(19) United States
(12) Patent Application Publication
(10) Pub. No.: US 2018/0164310 A1
(43) Pub. Date: Jun. 14, 2018

(54) DEVICE AND METHOD FOR IMMUNOCHROMATOGRAPHIC ASSAY

(71) Applicant: ABS ADVANCED BIOMEDICAL SYSTEMS S.R.L., Cernusco Sul Naviglio (IT)

(72) Inventor: Paolo GIORDANO, Cernusco Sul Naviglio (IT)

(21) Appl. No.: 15/837,625

(22) Filed: Dec. 11, 2017

Related U.S. Application Data
(63) Continuation of application No. PCT/IB2016/053114, filed on May 27, 2016.

(30) Foreign Application Priority Data
Jun. 10, 2015 (IT) 102015000022310

(51) Int. Cl. G01N 33/558

(52) U.S. Cl. CPC G01N 33/558

(57) ABSTRACT

A strip type device performs an immunochromatographic test and is used for diagnosis. The diagnostic test device determines the presence of an analyte in a sample, including a strip of material suitable for capillary migration of a liquid sample. The strip has a support on which are arranged and sequentially define different operative areas of the device according to a direction of capillary migration: a support portion for depositing the sample; a labeling portion including a first labeled reagent, recognizing the analyte to be determined or competing with the analyte; a chromatographic membrane on which are arranged in sequence and at a predetermined distance: a test line, on which a first capture reagent specific for the analyte and a second capture reagent specific for a second labeled reagent are immobilized; a control line on which a third capture reagent specific for the first labeled reagent is immobilized.

The present invention relates to a device for performing an immunochromatographic test, in particular a device of the strip type, and to a diagnostic method that uses such a device.

### PRIOR ART

Quick immunochromatographic tests, also known as lateral-flow immunoassay tests (LFIAS), can also be performed by untrained operators, while providing fast and reliable results in many areas, such as in the diagnosis of infectious or non-infectious diseases, in the use in emergency departments and in the defense against pathogens. In developing countries, the possibilities offered by LFIAS test can often represent the only opportunity for most of the population to receive a diagnosis and thus be treated accordingly. The utility of LFIAS tests is greater the higher the ability to determine the presence of analytes at low concentration.

In LFIAS tests, after adding the sample, the analyte to be determined and a suitable label are subjected to a chromatographic migration along a membrane and the result is read in the specific position of a capture ligand immobilized on the membrane itself. The most common technique involves the combined use of a colloidal gold-labeled ligand deposited, in dried form, on a support of material suitable for releasing the same labeled ligand and a medium capable of establishing a lateral flow on a nitrocellulose membrane. When there is a sufficient quantity of analyte in the sample, it forms an analyte-labeled ligand complex. Continuing the chromatographic migration, said complex encounters the capture ligand immobilized on the immunochromatographic membrane and thus a visible line forms.

Several approaches to increase the sensitivity of LFIAS tests have been published or patented.

In this context, reference is made to an LFIAS test of the “sandwich” type, in which the intensity of the test line is directly proportional to the concentration of the analyte in the sample being examined. In these tests, often used to detect the presence of an infectious antigen in the sample being examined, the increase in sensitivity allows decreasing the number of false negative responses of the tests.

In LFIAS tests of the “competitive” type, on the contrary, an increase in the concentration of the analyte of interest in the sample being examined causes the reduction in the intensity of the test line.

In order to increase the sensitivity of LFIAS tests, optoelectronic readers have been used, able to reveal the presence of a line in the position of the immobilized capture ligand even when the simple visual observation did not allow a safe identification of the line itself and thus the correct test interpretation.

However, the need for a reading device can be a decisive drawback when LFIAS tests are to be used in areas where electricity is not available or if the cost itself of the instrument is a limiting factor.

Many of the methods described to increase the sensitivity of LFIAS tests rely on modifications to the standard scheme, in terms of: a) internal or external amplification steps, with an increase in the number and/or complexity of the operative procedure steps; b) treatments of the samples on which the tests has to be carried out; c) other changes, such as to the structure and to the chromatographic features of the membranes.

Increases in sensitivity may also be obtained through the use of a scheme in which reagents are first mixed and then made to migrate on the chromatographic membrane. Among the reasons for using this scheme it is worth mentioning: i) the opportunity to avoid stability problems associated with the drying of reagents; ii) optimizing the sensitivity by reacting the various components in the liquid phase; or iii) possibility of using a microplate format. In the latter case, dried reagents into the wells can be preincubated for long times, in the order of tens of minutes and at temperatures above room temperature. The number of tests per unit time can still be high by processing multiple samples in parallel.

Beyond the higher complexity of the testing procedures, these methods however involve higher production costs which translate into higher costs for the end users and this might be a limiting factor for the spread in areas with underdeveloped economy.

A physical limit to the possibility of detection of LFIAS lines through visual observation is the limited ability of the human eye to identify the presence of a colloidal gold line or other colored labels, if the number of labeled particles is lower than a certain threshold. Moreover, the ability to identify the presence of a line is also influenced by the contrast of color/intensity of the line with respect to the bottom of the immunochromatographic membrane used in the test.

The sensitivity of an LFIAS test is, at least to a certain extent, proportional to the concentration of labeled particles and therefore a known method to increase sensitivity consists in increasing the optical density and/or the concentration of labeled particle to maximize the likelihood that the antigen-labeled particle complex forms and that said complex is captured by the immobilized line in test position. However, the major limitation of this approach is the possibility that non-specific bonds may occur, thus limiting the test specificity.

Typically, the human eye, in particular that of an untrained operator such as an operator that often uses LFIAS tests, is able to identify lines whose absorbance is greater than a threshold typically between 10 and 20 mAbs. For mAbs values, reference is made to the values read by the instrument Hamamatsu C10066 and by the program supplied with the same instrument for calculating the physical parameters of the line from the absorption profile graph. In particular, absorbance is measured as the logarithm of the ratio of the maximum reflectance (bottom line) to the minimum reflectance corresponding to the peak of maximum intensity of the colored line. Above this threshold, all operators are able to easily identify the presence of a line. Conversely, in the absorbance range of 10 and 20 mAbs (measured as above), only well-trained operators in the best conditions of observation are able to identify the presence of a line.

As a result, a very faint test line—such as a line of intensity between 4 and 10 mAbs although specific to the analyte of interest, and in the presence of a background of much lower intensity (0-2 mAbs), is not perceived by most of the operators.

U.S. Pat. No. 7,303,925 discloses a method for increasing the visual perception of a colored line in LFIAS tests, which uses the complementary colors to increase the contrast of the visual perception of the test result. An advantage is obtained in this way for the visual interpretation of a test result when the signal to be interpreted is visually weak due to the reduced amount of analyte. The invention provides a method for increasing the contrast that simplifies the visual perception of the color signal. In typical “sandwich” tests, in which a colored line indicates a positive signal, the color contrast method according to the invention helps decreasing the number of false negatives, especially for operators with visual defects of color perception.

U.S. Pat. No. 8,309,366 discloses methods and devices based on new lateral flow project schemes able to promote the interaction between ligands and specific markers, thereby allowing increasing the sensitivity in the detection of ligands of interest in the sample.

Anfossi et al. (Increased sensitivity of lateral flow immunoassay for ochratoxin A through silver enhancement, Anal Bioanal Chem. 2013, Vol. 405, pages 9859-9867) described a method to increase the sensitivity of LFIA tests using silver nucleation of colloidal gold, with a gain in sensitivity of more than 10 times compared to LFIAS tests based on colloidal gold alone.

Chunxiang Chen et al. (A Fast and Sensitive Quantitative Lateral Flow Immunoassay for CrylAb Based on a Novel Signal Amplification Conjugate, Sensors. 2012, 12, pages 11684-11696) described a new strategy for signal amplification in LFIAS tests, based on the amplification by a polylysine chain and the biotin-avidin amplification system.

However, all the methods described increase the complexity of the manufacturing procedures of LFIAS test and related costs and in some cases they also increase the complexity of the test run, another important parameter to allow use thereof by untrained operators.

Therefore, there is the need for a simple, cost-effective method which does not require instruments to increase the readability, and thus the sensitivity of LFIAS tests, minimizing the changes to both standard production procedures of LFIAS tests, in order to minimize the increase in costs, and to the execution procedure so as to facilitate the use thereof by untrained operators.

### DEFINITIONS

Test Line: Area of the immunochromatographic membrane in which the antibody, hereinafter referred to as Ligand 2, is immobilized, in which area the visible line forms whose presence (LFIAS direct tests) or disappearance (LFIAS competitive tests) provides the key to interpreting the test result. Normally, the Test Line is one, but as is known by the man skilled in the art, particularly in the case of semiquantitative LFIAS tests, the Test Lines can be more than one.

Control Line: Area of the immunochromatographic membrane in which the antibody, hereinafter referred to as Ligand 3, is immobilized, in which area the visible line forms due to the binding with all the labeled ligand, hereinafter referred to as Labeled ligand 1, which has passed the Test Line without binding to Ligand 2. The presence of the Control Line indicates that the different components of the test worked properly and that the chromatographic flow was sufficient to the appearance of the lines. Normally, the absence of the control line indicates an invalid test. Although normally present in the immunochromatographic tests for the above-mentioned reasons, the control line may however be not present or be made without the use of antibodies and immunological reactions.

Labeled ligand 1: a) Monoclonal or polyclonal antibody or antigen capable of binding to the analyte of interest or b) analyte of interest, labeled with colloidal gold or colored particles.

Ligand 2: Polyclonal or monoclonal antibody, or b) antigen capable of binding to the analyte of interest and immobilized in the “Test Line” zone of the immunochromatographic membrane.

Ligand 3: Polyclonal or monoclonal antibody able to bind to the Labeled ligand 1, in the prior art LFIAS tests immobilized in the “Control Line” zone of the immunochromatographic membrane.

### SUMMARY OF THE INVENTION

The immunochromatographic device and the method for its use according to the invention are outlined in the accompanying claims.

The present invention relates to a method that is simple and cost-effective at a production level and that does not introduce any operative step in the test run, to create a predetermined line, with absorbance intensity of about 10-15 mAbs (calculated as the logarithm of the ratio of the maximum reflectance to the minimum reflectance corresponding to the peak of maximum intensity of the colored line and measured by the instrument Hamamatsu C10066) in exactly the same position as the Test Line without, however, interfering with or being affected by the immunological antigen/antibody reaction and thereby without interfering with the test signal generation.

More in particular, the invention relates to a method for increasing the visibility of the Test Line in an immunochromatographic device which provides for overlapping, in the same Test Line, of Ligand 2 and a predetermined moiety of Ligand 3, so that the line produced by the labeled Ligand 1 with said moiety of Ligand 3 generates a bottom line whose intensity is equivalent or immediately below the perceptibility of the human eye and yet sufficiently intense to make the specific test line, generated by the interaction between the Labeled ligand 1, the analyte of interest and Ligand 2, visible, otherwise hardly perceptible by the human eye.

The same method can be usefully used, if in an immunochromatographic test of sandwich type it is necessary to improve the linearity of the test response for very low concentrations of the analyte of interest or, in competitive immunochromatographic tests, improve the linearity of the response for very high values of concentration of the analyte of interest.

The invention described provides a device and a method to improve the visual perceptibility of a line in an LFIAS test device by the superimposition of a bottom line adapted to increase the contrast between the test line and the bottom of the chromatographic membrane.

### BRIEF DESCRIPTION OF THE FIGURES

FIG. 1 schematically shows an immunochromatographic device according to the prior art, in which different materials are arranged on a support.

FIG. 2 schematically shows an immunochromatographic device according to the invention.

FIG. 3 shows a graph of the intensity of the Test Line as a function of the dilution factor of Ligand 3 (see definitions above).

FIG. 4 shows a graph of the intensity of the test line for a conventional LFIAS system of sandwich type (dotted line) and for an LFIAS system based on the present invention (solid line), for the detection of antigen DER P1, indicator of the presence of mites in house dust.

FIG. 5 shows a graph of the intensity of the test line for a standard LFIAS test (dotted line) and for an LFIAS test developed according to the present invention (solid line), for the detection of the presence of antigens Streptococcus A in throat swabs.

### DETAILED DESCRIPTION OF THE INVENTION

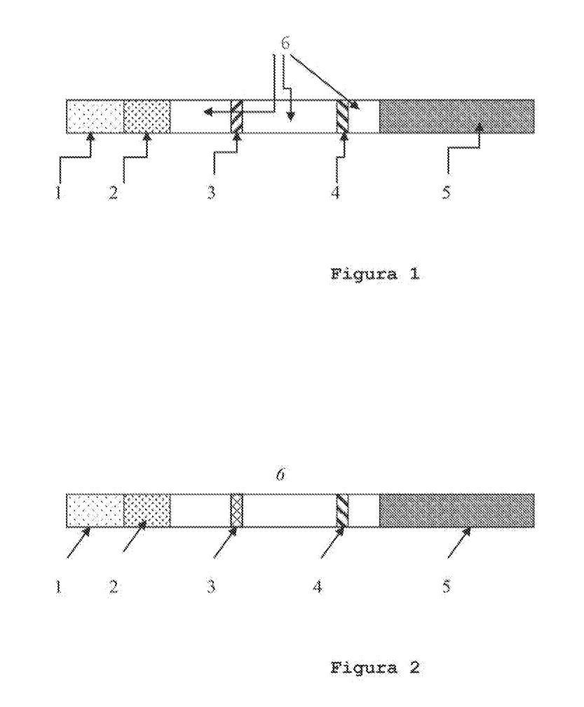

FIG. 1 schematically shows a device for immunochromatographic tests of the prior art.

On a support strip, typically a strip of plastic material, delimited portions are set up which define operative zones of the device in a sequence and which are as follows:

1=Support for sample deposition

2=Support for the Labeled ligand 1, directed against the analyte of which the presence or concentration is to be determined.

3=Test Line, obtained by immobilizing Ligand 2 on the immunochromatographic membrane, which recognizes the analyte itself.

4=Control Line, obtained by immobilizing Ligand 3 on the immunochromatographic membrane, which recognizes the Labeled ligand 1.

5=Absorbing pad to prevent the back-flow of the reaction fluid.

6=Chromatographic membrane.

In this example, a sample, or a liquid containing an analyte of interest, is applied on portion 1, in which the material receiving the sample has sufficient volume and porosity to receive and hold the liquid sample. The sample to be analyzed may be whole blood, serum or plasma, urine, extract from throat or vaginal swabs, or generally any biological fluid or any fluid in which an analyte to be determined is present in any form.

Specifically, as Labeled ligand 1 it is possible to use, by way of example, colloidal gold particles bound to an antibody or antigen.

The sample to be examined may be a liquid sample containing the analyte of interest. The term “analyte” or “analyte of interest” refers to the compound or mixture of compounds of which the presence or concentration has to be determined, which has at least one binding site or epitope. The analyte may be any substance for which a ligand exists or may be prepared. The analytes may be, without limitations, toxins, organic compounds, proteins, peptides, microorganisms, amino acids, nucleic acids, hormones, steroids, vitamins, drugs (both drugs administered for therapeutic use and those taken for illicit purposes), and metabolites or antibodies of any of the substances listed above. The term “analyte” includes any antigenic substance, haptens, antibodies, macromolecules and combinations thereof.

As said, a zone (portion 1) is found at one end (at the left end in the example) having sufficient volume and porosity to receive and hold the sample to be analyzed. The material receiving sample to be analyzed consists of any porous and absorbing material, such as paper, cellulose, cellulose derivatives such as cellulose acetate and nitrocellulose, glass fiber, fabrics, both natural such as cotton and synthetic (such as nylon), porous gels and so on.

The labeling portion2 comprises or is preferably made of glass fiber, but in certain embodiments it can be made of or comprise one of the materials used for the support portion 1.

In certain embodiments, the support portion 1 and the labeling portion 2 are made of the same material selected from paper, cellulose, cellulose derivatives such as cellulose acetate and nitrocellulose, glass fiber, fabrics, both natural such as cotton and synthetic (such as nylon), porous gels, and may consist of separate strips, partially overlapped, of such material or of a same strip of such material on which said portions 1 and 2 are identified.

The liquid sample migrates by capillary action and as soon as the sample enters portion 2 which contains the Labeled ligand 1 against the analyte to be determined, the Labeled ligand 1 is put in suspension in the liquid sample, according to a process well known to the man skilled in the art.

If the antigen is present in the sample, a complex forms with the Labeled ligand 1 and said complex migrates until it reaches portion 3. In zone 3, the analyte-labeled ligand 1 complex binds to Ligand 2 immobilized in portion 3, thus giving rise to a visible line, if the number of particles of colloidal gold or other material is high enough to generate a visible line.

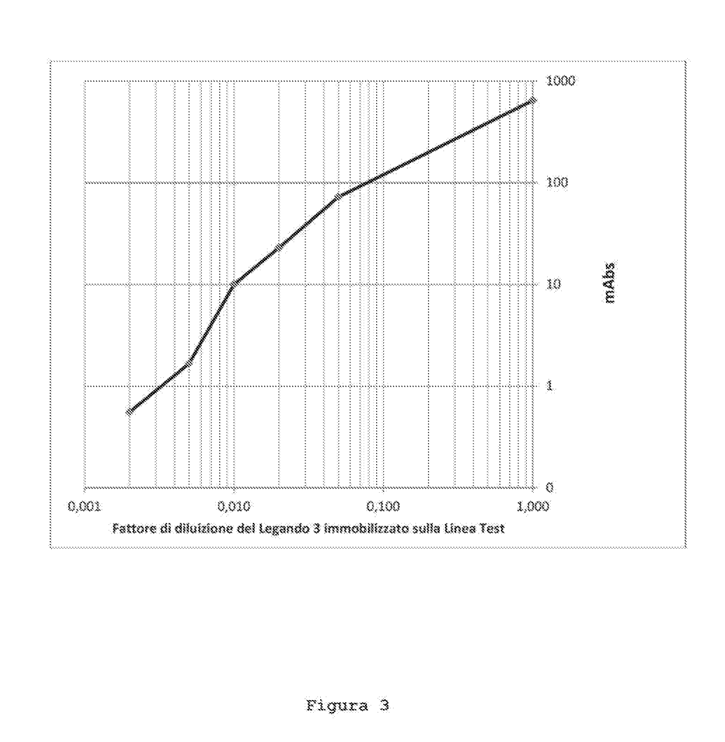

FIG. 2 instead shows a device according to the present invention in which, similarly to the devices of the prior art, delimited portions are prepared on a support strip, typically a plastic strip, which define in sequence different operative areas of the device and which are, however, as follows:

1=support portion for sample deposition

2=labeling portion with the Labeled ligand 1, able to recognize the analyte to be determined

3=Test Line, obtained by immobilizing a mixture of Ligand 2 able to recognize the same analyte and a predetermined amount of Ligand 3 on the immunochromatographic membrane.

4=Control line, obtained by immobilizing Ligand 3 able to recognize the Labeled ligand 1 on the immunochromatographic membrane.

5=absorbing portion to prevent the back-flow of the reaction fluid.

6=Chromatographic membrane.

The difference between the prior art device and that of the invention is therefore that a predetermined concentration of Ligand 3, that is, of the antibody normally used in the Control Line, is immobilized on the Test Line 3 together with Ligand 2 able to recognize the analyte to be determined.

In this way, a line of particles of Labeled ligand 1 (for example, colloidal gold) forms even if the sample does not contain the analyte of interest. By accurately adapting the concentration of Ligand 3 in portion 3, it is easily possible to generate a line whose intensity is at a similar level or immediately below the threshold of visibility to the human eye.

If the sample contained an analyte concentration below the limit of sensitivity of the system, that is, if the sample per se generated a non-visible line in zone 2, the overlap of the two populations of particles of Labeled ligand 1—that is, a) particles immobilized by Ligand 3 and b) particles immobilized by Ligand 2—it would give rise to a visible line, thus increasing the sensitivity of the test.

The predetermined concentration of Ligand 3 present on the Test Line 3 together with Ligand 2 is between 1:1 and 1:500 with respect to the concentration of Ligand 3 used in the control line 4, so as to reach an absorbance intensity equivalent to or immediately below the threshold of visibility to the human eye, i.e. of 10-15 mAbs, measured with the instrument Hamamatsu 10066 already described.

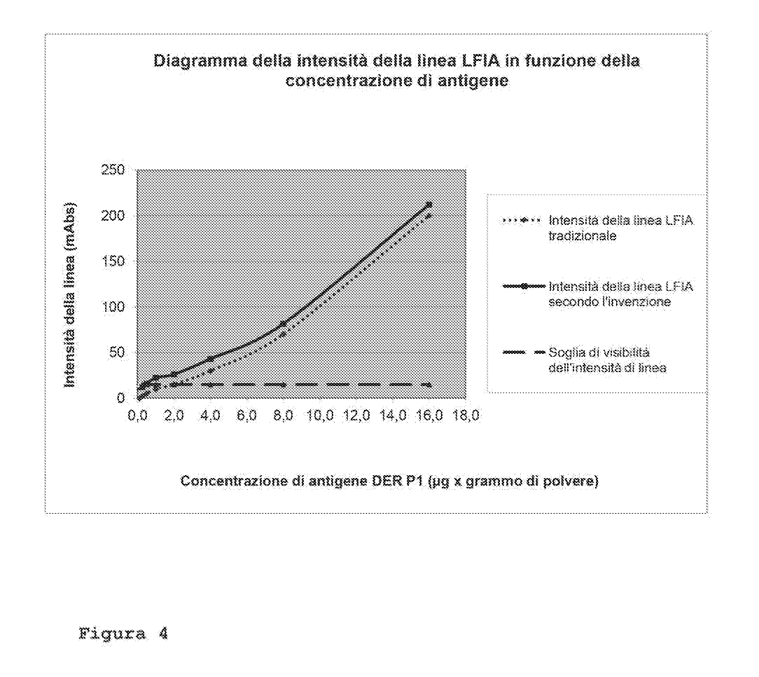

FIG. 3 shows the graph of the intensity of the Test Line as a function of the dilution factor of Ligand 3. In the example shown, a dilution factor of 1:100 with respect to the concentration of Ligand 3 used for the Control Line, immobilized in the test line position, gives rise to a line—as determined by the instrument Hamamatsu already described—of about 10 mAbs if the analyte to be determined is not present in the sample.

The invention will be further described by means of the following embodiment examples.

### EXAMPLE 1

In a first example, the sample is represented by an aqueous buffer in which a quantity of house dust is dispersed, in which the presence of mite feces is to be determined. Said sample is applied to portion 1.

The Labeled ligand 1, in particular a rabbit polyclonal antibody anti-DER P1, conjugated with colloidal gold particles of average diameter of 40 nm, is deposited on portion 2.

Two different lines at 5 mm distance from each other are dispensed on the immunochromatographic membrane.

A solution in distilled water or in a suitable buffer of a rabbit polyclonal antibody anti-DER P1 and of a goat anti-rabbit IgG antibody is dispensed on the Test Line (portion 3), respectively in concentrations of from 0.01 to 1.1 μg of antibody per mm of membrane and from 0.1 to 100 ng of antibody per mm of membrane.

A solution of goat anti-rabbit IgG antibody in distilled water or in an appropriate buffer is released on the control line (portion 4), at a concentration of from 0.01 to 50 μg per mm of membrane.

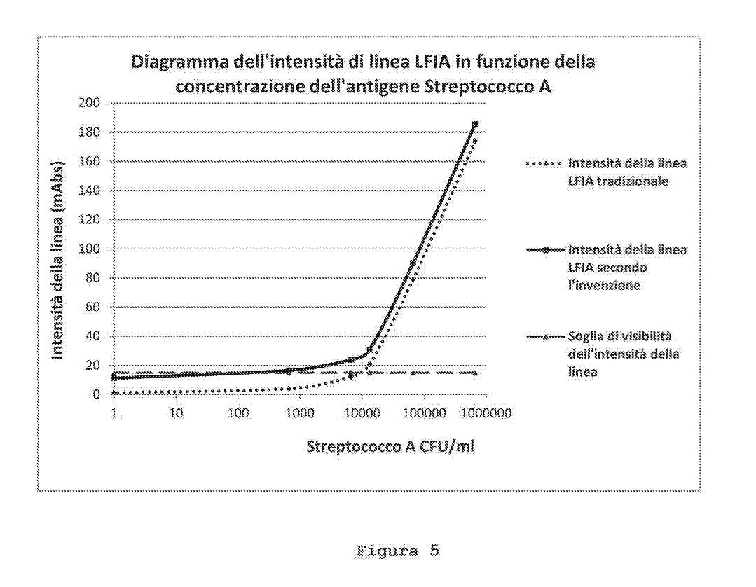

FIG. 4 shows the graph of the intensity of the test line for a traditional LFIA system (dotted line) and for an LFIA system based on the present invention (solid line). The dashed line represents the intensity of the line corresponding to the minimum level of human eye perception.

Concentrations of mite feces below 2 μg per gram of dust are the lower limit of visibility for a standard LFIA test, while concentrations of 0.5 μg per g of dust give rise to a clearly visible line in the LFIA tests in example 1 produced according to the present invention.

### EXAMPLE 2

In a second example, the sample is a pharyngeal swab in which the presence of Streptococcus pyogenes antigens of group A is to be determined. The antigen is extracted by means of one of several methods, for example by the use of nitrous acid, hydrochloric acid, etc. and then the extracted liquid is applied on portion 1.

The Labeled ligand 1 is deposited on portion 2, in particular a rabbit anti-Strep A polyclonal antibody, conjugated with colloidal gold particles having an average diameter of 40 nm.

Two different lines at 5 mm distance from each other are dispensed on the immunochromatographic membrane.

A solution in distilled water or in a suitable buffer of a rabbit anti-Strep A polyclonal antibody and of a goat anti-rabbit IgG antibody is dispensed on the Test Line (portion 3), respectively in concentrations of from 0.01 to 1.1 μg of antibody per mm of membrane and from 0.1 to 100 ng of antibody per mm of membrane.

A solution of goat anti-rabbit IgG antibody in distilled water or in an appropriate buffer is released on the control line (portion 4), at a concentration of from 0.01 to 50 μg per mm of membrane.

FIG. 5 shows the graph of the intensity of the test line for a standard LFIA test (dotted line) and for an LFIA test developed according to the present invention (solid line). The dashed line represents the intensity of the line corresponding to the minimum level of intensity that can be perceived by the human eye under normal conditions.

Concentrations of Strep A below 1.3×10⁴ CFU/mL are unlikely to be viewed using standard LFIA tests, while concentrations of Strep A of 6.6×10³ CFU/mL are clearly visible with LFIA tests prepared according to the present invention.

While a method has been described for increasing the visibility of test lines using a predetermined concentration of the same antibody used for capturing the labeled primary antibody and forming the Control Line, the invention described is not limited to this embodiment but includes the use of any set of capturing agents capable of generating lines whose intensity can be close to the limit of visibility. By way of example, such a line could be generated, in the same position as the test line, by depositing a predetermined concentration of an antibody capable of recognizing a labeled complex not interfering with the analyte-labeled ligand conjugation process. Alternatively, said line may be generated by any analyte recognized by a labeled antibody. The labeling agents may be any type of particle used in the various types of immunochromatographic methods, such as colloidal gold particles, colored particles, etc. However, the use of the same Labeled ligand 1 used in the formation reaction of the test line bound to the same Ligand 3 used for forming the control line allows considerably simplifying the production process, thereby reducing the cost thereof.

Moreover, the method reported, in addition to the increase of qualitative and semiquantitative LFIA test sensitivity, also allows improving the linearity of the response curve of the test for very low concentration values of the analyte of interest (sandwich tests) or for very high concentration values of the analyte (competitive tests).

It is clear that only some particular embodiments of the present invention have been described, and those skilled in the art will be able to make all the necessary modifications for its adaptation to particular applications, without departing from the protection scope of the present invention.

1. Diagnostic test device for determining presence of an analyte in a sample, comprising a strip comprising a material suitable for capillary migration of a liquid sample, said strip comprising a support on which the following delimited portions are arranged, which sequentially define different operative areas of the test device, according to a direction of propagation by capillary action of the liquid sample:
a support portion for depositing the sample;
a labeling portion comprising a first labeled reagent, able to recognize an analyte to be determined;
a chromatographic membrane on which the following are arranged in sequence and at a predetermined distance:
i) at least one test line, on which a first capture reagent specific for said analyte and a predetermined amount of a second capture reagent specific for a second labeled reagent equal to or different from said first labeled reagent are immobilized.

2. Device according to claim 1, wherein the chromatographic membrane comprises at least one control line on which a third capture reagent specific for said first labeled reagent is immobilized.

3. Device according to claim 1, wherein said second capture reagent is equal to said third capture reagent and wherein said first labeled reagent is equal to said second labeled reagent.

4. Device according to claim 1, wherein said strip comprises an absorbing portion arranged in succession with respect to said at least one control line, along the direction of propagation of the liquid sample by capillary action.

5. Device according to claim 1, wherein said liquid sample is selected from a whole blood sample, serum or plasma, urine, throat or vaginal swab extract, and a biological fluid or non-biological fluid in which an analyte to be determined, in any form, is present.

6. Device according to claim 1, wherein said first and/or second labeled reagent is selected from: a) a monoclonal or polyclonal antibody or an antigen capable of binding to the analyte of interest or b) an analyte of interest, labeled with colloidal gold or colored particles.

7. Device according to claim 1, wherein said analyte to be determined is selected from toxins, organic compounds, proteins, peptides, microorganisms, amino acids, nucleic acids, hormones, steroids, vitamins, medications, drugs and their metabolites or antibodies.

8. Device according to claim 1, wherein said support portion comprises a material selected from paper, cellulose, cellulose derivatives such as cellulose acetate and nitrocellulose, glass fiber, fabrics, both natural cotton and synthetic nylon and porous gels.

9. Device according to claim 1, wherein said labeling portion comprises of glass fiber or a material selected from paper, cellulose, cellulose derivatives such as cellulose acetate and nitrocellulose, glass fiber, fabrics, both natural cotton and synthetic nylon and porous gels.

10. Device according to claim 1, wherein said predetermined amount of said second capture reagent specific for said second labeled reagent equal to or different from said first labeled reagent in said test line is at a level such as to generate a line with intensity equivalent to, or immediately below a threshold of visibility for the human eye of a complex between said second capture reagent and said second labeled reagent in the absence of said analyte to be determined.

11. Device according to claim 10, wherein said predetermined amount of said second capture reagent is comprised between 1:1 and 1:500 with respect to the concentration of said second capture reagent, or alternatively of said third capture reagent, used in the control line, so as to achieve an absorbance intensity equivalent to, or immediately below 10-15 mAbs, as measured by the instrument Hamamatsu C10066.

12. Device according to claim 1, wherein said first capture reagent is selected from: a) a monoclonal or polyclonal antibody or b) an antigen capable of binding to the analyte of interest.

13. Device according to claim 1, wherein said second capture reagent is selected from: a) a monoclonal or polyclonal antibody capable of binding to said first labeled reagent, b) an antibody capable of recognizing a labeled complex not interfering with the analyte-labeled ligand conjugation process, and c) any analyte recognized by a labeled antibody; and wherein said third capture reagent is a monoclonal or polyclonal antibody capable of binding to said first labeled reagent.

14. Method for determining presence of an analyte to be determined in a sample, comprising the following steps:
1) providing a device as defined in claim 1;
2) depositing a liquid sample containing said analyte to be determined on the support portion of said device;
3) eluting the liquid sample on said device until reaching said absorbing portion
4) in a sandwich test, detecting formation of a colored line at said test line, wherein the formation of said colored line indicates the presence of said analyte to be determined; or
5) in a competitive test, detecting discoloration of a colored line at said test line, wherein said discoloration of the colored line indicates the presence of the analyte to be determined.

15. Method according to claim 14, comprising the step of detecting the formation of a colored line at said control line, wherein the formation of such colored line indicates that the test has been performed correctly.
